(19) United States
(12) Patent Application Publication
(10) Pub. No.: US 2015/0056686 A1
(43) Pub. Date: Feb. 26, 2015

(54) GAS-BORNE PATHOGEN SENSOR APPARATUSES AND METHODS THEREOF

(71) Applicant: Nth Tech Corporation, Churchville, NY (US)

(72) Inventor: Michael D. Potter, Churchville, NY (US)

(21) Appl. No.: 14/463,819

(22) Filed: Aug. 20, 2014

Related U.S. Application Data
(60) Provisional application No. 61/867,765, filed on Aug. 20, 2013.

(51) Int. Cl. C12Q 1/04

(52) U.S. Cl. CPC C12Q 1/04
USPC 435/287.1; 29/428

(57) ABSTRACT

A gas-borne pathogen sensor apparatus includes a chamber in a housing having a gas inlet, a gas outlet, and a collection area with a fluid exit port, a liquid misting device, and a sensor element. The liquid misting device is coupled to the chamber and is positioned to spray fluid into the chamber in the housing. The sensor element is positioned in the chamber in the housing and has one or more probes designed to mate with any of one or more targets in a gas supplied to the chamber in the housing through the gas inlet.

This application claims the benefit of U.S. Provisional Patent Application Ser. No. 61/867,765 filed on Aug. 20, 2013, which is hereby incorporated by reference in its entirety.

### FIELD

This technology generally relates to devices and methods for sensing particles and, more particularly, to gas-borne pathogen sensor apparatuses and methods thereof.

### BACKGROUND

Viable micro organisms in an aqueous environment have been shown to have a very strong dipole moment. This leads to an extraordinarily large frequency dependent relative permittivity. The existence of this extraordinarily large frequency dependent relative permittivity has been used by sensor apparatuses and sensor arrays for the detection of water borne microbial pathogens, such as U.S. Pat. No. 8,373,206, which is herein incorporated by reference in its entirety.

However, many pathogenic microbes are not water borne, but instead are air-borne. Illustrative examples of air-borne microbes include Bacillus anthracis spores, long surviving bacteria, protozoa, various varieties of viruses, including dormant phase viruses, bacteria, and protozoa. Unfortunately, effective sensor apparatuses and methods for the rapid detection of air-borne microbial pathogens currently do not exist.

### SUMMARY

A gas-borne pathogen sensor apparatus includes a housing defining a chamber having at least a liquid inlet and a gas inlet gas inlet, a gas permeable membrane and a sensor element. The gas permeable membrane is positioned across the gas inlet and is configured to retain liquid in the chamber and to allow a gas to enter into the chamber. The sensor element is positioned in at least one of the chamber of the housing or the housing outlet and has one or more probes designed to mate with any one or more targets in the gas supplied to the chamber through the gas inlet.

A method for making a gas-borne pathogen sensor apparatus includes providing a housing defining a chamber having at least a liquid inlet and a gas inlet, a gas permeable membrane and a sensor element. A gas permeable membrane is positioned across the gas inlet and is configured to retain liquid in the chamber and to allow a gas to enter into the chamber. A sensor element is positioned in at least one of the chamber of the housing or the housing outlet and has one or more probes designed to mate with any one or more targets in the gas supplied to the chamber through the gas inlet.

A gas-borne pathogen sensor apparatus includes a chamber in a housing having a gas inlet, a gas outlet, and a collection area with a fluid exit port, a liquid misting device, and a sensor element. The liquid misting device is coupled to the chamber and is positioned to spray fluid into the chamber in the housing. The sensor element is positioned in the chamber in the housing and has one or more probes designed to mate with any of one or more targets in a gas supplied to the chamber in the housing through the gas inlet.

A method for making a gas-borne pathogen sensor apparatus includes providing a chamber in a housing having a gas inlet, a gas outlet, and a collection area with a fluid exit port. A liquid misting device is coupled to the chamber in the housing and is positioned to spray liquid into the chamber in the housing. A sensor element is positioned in the chamber of the housing and has one or more probes designed to mate with any of one or more targets in a gas supplied to the chamber in the housing through the gas inlet.

This technology provides a number of advantages including providing new sensor apparatuses and methods for rapid detection of gas-borne microbial pathogens or other targets. With this technology, gas-borne particles, such as microbial pathogens and microbes in general, chemical agents, explosive materials, and other targets can easily and effectively be identified. This technology may include various approaches for agitation or mixing in order to enhance transfer of pathogens or particles to the water or other fluids in the chamber of the sensing device.

### BRIEF DESCRIPTION OF THE DRAWINGS

FIG. 1 is a diagram of an example of a sensor apparatus with a sensor element located in a main body of a water chamber;

FIG. 2 is a diagram of an example of another sensor apparatus with a sensor element located outside a main body of a water chamber; and

FIG. 3 is a diagram of an example of yet another sensor apparatus.

### DETAILED DESCRIPTION

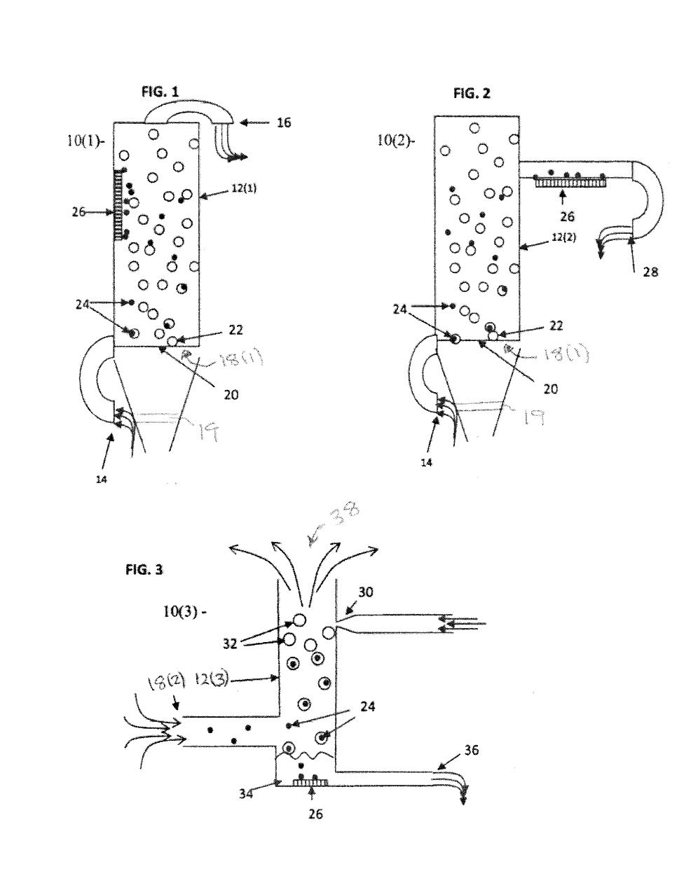

Examples of real-time sensor apparatuses 10(1)-10(3) for the rapid detection of gas-borne microbial pathogens or other airborne targets are illustrated in FIGS. 1-3. Each of these real-time sensor apparatuses 10(1)-10(3) includes one of the chambers 12(1), 12(2), or 12(3), one of the liquid inlet 14 or liquid misting device 30, one of the gas inlets 18(1) or 18(2), one of the housing outlets 16, 28, or 38, and at least one of the sensor elements 26, although each of these apparatuses can include other types and/or numbers of other systems, devices, components, and/or other elements in other configurations as illustrated and described with the examples herein. This technology provides a number of advantages including providing new sensor apparatuses and methods for rapid detection of gas-borne microbial pathogens or other targets.

Referring more specifically to FIG. 1, the sensor apparatus 10(1) for the rapid detection of gas-borne microbial pathogens or other airborne targets in accordance with one example is illustrated. In this particular example, the sensor apparatus 10(1) includes the housing 12(1) which defines a chamber, a gas inlet 18(1), and the sensor element 26, although the sensor apparatus 10(1) could include other types and numbers of systems, devices, components and/or other elements in other configurations as illustrated and described herein.

The housing 12(1) includes the liquid inlet 14, such as an inlet tube by way of example only, the housing outlet 16, such as an outlet tube by way of example only, and a gas permeable membrane 20, although the housing 12(1) can have other types and numbers of systems, device, components and/or other elements in other configurations. In this particular example, the liquid inlet 14 is adjacent to one end of the housing 12(1) and the housing outlet 16 is spaced from the liquid inlet 14 and both provide access for fluids to enter and exit the chamber in the housing 12(1). The gas permeable membrane 20 provides a liquid seal at one end of the chamber in the housing 12(1) adjacent the liquid inlet 14. The gas permeable membrane 20 permits air or another gas which may have one or more targets to pass through into the chamber in the housing 12(1) while retaining any liquid, such as water, in the chamber in the housing 12(1), although other types of air or other liquid inlet systems could be used.

A forced gas device 19, such as a fan or compressor by way of example only, may be coupled to the gas inlet 18(1) to the chamber in the housing 12(1) about at least a portion of the gas permeable membrane 20. When the forced gas device is engaged, air or another gas which may have the one or more targets is forced or otherwise directed against the membrane 20, although other manners for providing air or another gas to the chamber in the housing 12(1) could be used, such as natural drift of the air or another gas into the chamber in the housing 12(1).

The sensor element 26 is located in the chamber in the housing 12(1), although the sensor element 26 could be in other locations and other numbers of sensor elements could be used. The sensor element 26 may have one or more probes configured to detect one or more targets, such as with high specificity molecular probes or other suitable probes. By way of example only, a sensor element 26, such as the one illustrated and described in U.S. Pat. No. 8,373,206, which has been incorporated by reference in its entirety, may be used.

An example of the operation of the sensor apparatus 10(1) will now be described with reference to FIG. 1. When the sensor apparatus 10(1) is engaged, water or another liquid, is continuously supplied to the chamber in the housing 12(1) via the liquid inlet 14 to fill the chamber in the housing 12(1) above the sensor element 26 and exit through the housing outlet 16, although the chamber in the housing 12(1) could be filled with liquid to other levels and in other manners, such as to a fixed level in the chamber in the housing 12(1) by way of example only. The gas permeable membrane 20 provides a liquid seal to retain any liquid, such as water, in the chamber in the housing 12(1).

When engaged, the forced gas device 19 drives air or another gas which may have the one or more targets against and through the gas permeable membrane 20 into the liquid in the chamber in the housing 12(1). In this particular example, as the air or other gas to be tested is forced through the gas permeable membrane 20 into the liquid in the chamber in the housing 12(1) small bubbles 22 of the air or other gas are formed in the liquid in the chamber in the housing 12(1). Eventually particles or other targets 24, such as contaminants, in the air or gas are released from the bubbles 22 effectively becoming water-borne or fluid-borne particles or other targets, within the liquid in the chamber in the housing 12(1). Meanwhile the air or other gas introduced into the chamber in the housing 12(1) by the forced gas device 19 may eventually exit through the housing outlet 16.

When a target, such as a contaminant or other particle, becomes released in the liquid in the chamber in the housing 12(1) and is a match with one or more probes of the sensor element 26, then the target can mate or otherwise attach to the one or more probes on the sensor element 26. When a positive detection is indicated by the sensor element 26, this positive detection can for example be output, such as with a display (not shown) on the sensor element 26 or to another monitoring computing device (not shown) which is coupled to and is monitoring output signals from the sensor element 26, although other approaches for signaling a detection can be used.

Referring to FIG. 2, the sensor apparatus 10(2) for the rapid detection of gas-borne microbial pathogens or other airborne targets in accordance with another example is illustrated. Elements in sensor apparatus 10(2) which are like those in sensor apparatus 10(1) will have like reference numerals. The structure and operation of the sensor apparatus 10(2) is the same as the sensor apparatus 10(1), except as illustrated and described herein.

The housing 12(2) includes the liquid inlet 14, such as an inlet tube by way of example only, the housing outlet 28, such as an outlet tube by way of example only, and the gas permeable membrane 20, although the housing 12(2) can have other types and numbers of systems, device, components and/or other elements in other configurations. In this particular example, the liquid inlet 14 is adjacent one end of the housing 12(2) and the housing outlet 28 is spaced from the liquid inlet 14 and both provide access for fluids to enter and exit the chamber in the housing 12(2). The gas permeable membrane 20 provides a liquid seal at one end of the chamber in the housing 12(2) adjacent the liquid inlet 14. The gas permeable membrane 20 permits air or another gas which may have one or more targets to pass through into the chamber in the housing 12(2) while retaining any liquid, such as water, in the chamber in the housing 12(2), although other types of air or other liquid inlet systems could be used.

A forced gas device 19, such as a fan or compressor by way of example only, may be coupled to the gas inlet 18(1) chamber in the housing 12(2) about at least a portion of the gas permeable membrane 20. When the forced gas device 19 is engaged, air or another gas which may have the one or more targets is forced or otherwise directed against the membrane 20, although other manners for providing air or another gas to the chamber in the housing 12(2) could be used.

The sensor element 26 is located outside the chamber in the housing 12(2) in the housing outlet 28 comprising an outlet tube in this example, although the sensor element could be in other locations and other numbers of sensor elements could be used. The sensor element 26 may have one or more probes configured to detect one or more targets, such as with high specificity molecular probes or other suitable probes. By way of example only, a sensor element 26, such as the one illustrated and described in U.S. Pat. No. 8,373,206, which has been incorporated by reference in its entirety, may be used.

An example of the operation of the sensor apparatus 10(2) will now be described with reference to FIG. 2. When the sensor apparatus 10(2) is engaged, water or another liquid, is continuously supplied to the chamber in the housing 12(2) via liquid inlet 14 and may exit through the housing outlet 28 passing the sensor element 26 located in the housing outlet 28 in this example, although the chamber in the housing 12(1) could be filled with liquid in other manners and the sensor element could be in other locations. The gas permeable membrane 20 provides a liquid seal to retain any liquid, such as water, in the chamber in the housing 12(2).

When engaged, the forced gas device 19 drives air or another gas along with any targets which may be in the air or another gas against and through the gas permeable membrane 20 into the liquid in the chamber in the housing 12(2). In this particular example, as the air or other gas to be tested is forced through the gas permeable membrane 20 into the liquid in the chamber in the housing 12(2) small bubbles 22 of the air or other gas are formed in the liquid in the chamber in the housing 12(2). Eventually particles or other targets 24, such as contaminants, in the air or gas are released from the bubbles 22 effectively becoming water-borne or fluid-borne particles or other targets, within the liquid in the chamber in the housing 12(2). Meanwhile the air or other gas introduced into the chamber in the housing 12(2) by the forced gas device 19 may eventually exit through the housing outlet 28.

When a target, such as a contaminant or other particle, released in the liquid in the chamber in the housing 12(2) is a match with one or more probes of the sensor element 26 in the housing outlet 28, then the target can mate or otherwise attach to the one or more probes on the sensor element 26. When a positive detection is indicated by the sensor element 26, this positive detection can for example be output, such as with a display (not shown) on the sensor element 26 or to another monitoring computing device (not shown) which is coupled to and is monitoring output signals from the sensor element 26, although other approaches for signaling a detection can be used.

Referring to FIG. 3, the sensor apparatus 10(3) for the rapid detection of gas-borne microbial pathogens or other airborne targets in accordance with yet another example is illustrated. Elements in sensor apparatus 10(3) which are like those in sensor apparatus 10(1) will have like reference numerals. The structure and operation of the sensor apparatus 10(3) is the same as the sensor apparatus 10(1), except as illustrated and described herein.

In this particular example, the sensor apparatus 10(3) includes a housing 12(3) which defines a chamber, a liquid misting device 30, and a sensor element 26, although the sensor apparatus 10(3) could include other types and numbers of systems, devices, components or other elements in other configurations as illustrated and described herein.

The housing 12(3) includes a chamber with a gas inlet 18(2), a housing outlet 38, and a collection area 34 with an exit port 36, although the housing 12(3) could have other types of inlets, outlets, components, and/or other elements in other configurations. The gas inlet 18(2) in this example comprises an inlet tube at one side of the housing 12(3) and the housing outlet 38 is spaced from the gas inlet 18(2). Both the gas inlet 18(2) and housing outlet 38 enable air or other gases to enter and exit the chamber in the housing 12(3). The collection area 34 is positioned below a liquid misting device 30, although the collection area could be in other locations. The collection area 34 includes the sensor element 26 and the exit port 36 to provide space for collecting the mist with any targets which may be in the air or another gas for detection by the sensor element 26 and for the collected mist to eventually exit the collection area 34, although other arrangements could be used.

A liquid misting device 30 is connected to the chamber in the housing 12(3) adjacent the housing outlet 38, although the liquid misting device 30 could be connected in other locations and other types and numbers of fluid misting devices or other fluid supply devices could be used in other configurations. The liquid misting device 30 is configured to be coupled to a water source or other liquid source (not shown) and to spray a liquid mist into the chamber of the housing 12(3) when engaged.

The sensor element 26 is located in the collection area 34 of the chamber in the housing 12(3), although the sensor element 26 could be in other locations and other types and/or numbers of sensor elements could be used. The sensor element 26 is configured to detect one or more targets, such as with high specificity molecular probes or other suitable probes. By way of example only, a sensor element such as the one illustrated and described in U.S. Pat. No. 8,373,206, which has been incorporated by reference in its entirety, may be used.

An example of the operation of the sensor apparatus 10(3) will now be described with reference to FIG. 3. When the sensor apparatus 10(3) is engaged, air or another gas which may contain one or more targets enters into the chamber in the housing 12(3) via the gas inlet 18(2). The air or gas may enter by natural drift or by a forced gas device (not shown) which has been engaged. The liquid misting device 30 is engaged to spray water or other liquid mist into the chamber of the housing 12(3). As the liquid mist is introduced into the chamber of the housing 12(3), particles 24, such as microbes or other targets by way of example only, are washed from the air or other gas by the water droplets 32.

The water or other liquid droplets 32 with any targets which may be in the introduced air or other gas are collected in the collection area 34 of chamber in the housing 12(3). When a target in the collected water or other liquid in the collection area 34 is a match with one or more probes of the sensor element 26 in the collection area 34, then the target can mate or otherwise attach to the one or more probes on the sensor element 26. When a positive detection is indicated by the sensor element 26, this positive detection can for example be output, such as with a display (not shown) on the sensor element 26 or to another monitoring computing device (not shown) which is coupled to and is monitoring output signals from the sensor element 26, although other approaches for signaling a detection can be used. Meanwhile excess water or other collected liquids can exit from the collection area 34 of the chamber of the housing 12(3) through the exit port 36.

Accordingly as illustrated and described with the examples herein, this technology provides new sensor apparatuses and methods for rapid detection of gas-borne microbial pathogens or other targets. With this technology, real-time continuous sensor monitoring of target gas-borne microbes or particles is provided.

Having thus described the basic concept of the invention, it will be rather apparent to those skilled in the art that the foregoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and scope of the invention. Additionally, the recited order of processing elements or sequences, or the use of numbers, letters, or other designations therefore, is not intended to limit the claimed processes to any order except as may be specified in the claims. Accordingly, the invention is limited only by the following claims and equivalents thereto.

What is claimed is:

1. A gas-borne pathogen sensor apparatus comprising:
a housing defining a chamber and having at least a liquid inlet and a gas inlet;
a gas permeable membrane positioned across the gas inlet, the gas permeable membrane is configured to retain liquid in the chamber and to allow a gas to enter into the chamber; and
a sensor element positioned in at least one of the chamber of the housing or the housing outlet, the sensor element comprising one or more probes designed to mate with any one or more targets in the gas supplied to the chamber through the gas inlet.

2. The device as set forth in claim 1 further comprising a forced gas device coupled to the gas inlet.

3. The device as set forth in claim 1 wherein the sensor element is positioned in the chamber of the housing.

4. The device as set forth in claim 1 wherein the housing outlet comprises a housing outlet tube and the sensor element is positioned in housing outlet tube.

5. A method for making a gas-borne pathogen sensor apparatus, the method comprising:
providing a housing defining a chamber and having at least a liquid inlet and a gas inlet;
positioning a gas permeable membrane across the gas inlet, the gas permeable membrane is configured to retain liquid in the chamber and to allow a gas to enter into the chamber; and
positioning a sensor element in at least one of the chamber of the housing or the housing outlet, the sensor element comprising one or more probes designed to mate with any one or more targets in the gas supplied to the chamber through the gas inlet.

6. The method as set forth in claim 5 further comprising coupling a forced gas device to the gas inlet.

7. The method as set forth in claim 5 wherein the positioning the sensor element further comprises positioning the sensor element in the chamber of the housing.

8. The method as set forth in claim 5 wherein the housing outlet comprises a housing outlet tube and wherein the positioning the sensor element further comprises positioning the sensor element in the housing outlet tube.

9. A gas-borne pathogen sensor apparatus comprising:
a chamber in a housing having a gas inlet, a gas outlet, and a collection area with a fluid exit port;
a liquid misting device coupled to the chamber, the liquid misting device positioned to spray fluid into the chamber in the housing;
a sensor element positioned in the chamber in the housing, the sensor element comprising one or more probes designed to mate with any of one or more targets in a gas supplied to the chamber in the housing through the gas inlet.

10. The device as set forth in claim 9 wherein the sensor element is in the collection area in the chamber in the housing.

11. A method for making a gas-borne pathogen sensor apparatus, the method comprising:
providing a chamber in a housing having a gas inlet, a gas outlet, and a collection area with a fluid exit port;
coupling a liquid misting device to the chamber in the housing, the liquid misting device positioned to spray liquid into the chamber in the housing;
positioning a sensor element in the chamber of the housing, the sensor element comprising one or more probes designed to mate with any of one or more targets in a gas supplied to the chamber in the housing through the gas inlet.

12. The device as set forth in claim 11 wherein the positioning the sensor element further comprises positioning the sensor element is in the collection area in the chamber in the housing.
